(19) United States
(12) Patent Application Publication
(10) Pub. No.: US 2013/0239339 A1
(43) Pub. Date: Sep. 19, 2013

(54) COMPOSITION AND METHOD

(71) Applicants: Gavin Bown, Saint Germain en Laye (FR); Carl Clayton, East Yorkshire (GB); Malcolm McKechnie, East Yorkshire (GB); James Young, Dubai (AE)

(72) Inventors: Gavin Bown, Saint Germain en Laye (FR); Carl Clayton, East Yorkshire (GB); Malcolm McKechnie, East Yorkshire (GB); James Young, Dubai (AE)

(73) Assignee: RECKITT & COLMAN (OVERSEAS) LIMITED, Berkshire (GB)

(21) Appl. No.: 13/698,945

(22) PCT Filed: May 12, 2011
(86) PCT No.: PCT/GB2011/050914

§ 371 Date: Dec. 3, 2012

(30) Foreign Application Priority Data
May 20, 2010 (GB) 1008364.0

(51) Int. Cl. A61K 8/14; A61Q 9/04

(52) U.S. Cl. CPC A61K 8/14; A61Q 9/04
USPC 8/161

(57) ABSTRACT

A method of delivering a primary active agent and a secondary active agent to a locus. The method uses a polymersome containing composition. The composition comprises a plurality of polymersome vesicles containing the secondary active agent and a liquid matrix comprising the primary active agent. The polymersome vesicles are capable of being disrupted by the application of mechanical shear.

The present invention relates to a method of delivering a plurality of active agents to a locus and to a composition for use in same.

Consumers are aware that certain household compositions require multiple individual actives to achieve their aim.

For example in the field of cleaning it is recognised that in order to provide sufficient efficacy a multitude of different cleaning agents have to be incorporated into a single cleaning composition.

As examples bleaches are used to oxidise/decolourise stains; surfactants are used to solubilise grease and water softening agents are used to soften hard water. Auxiliary agents may also be required to raise the activity of some actives.

One major problem with the preparation of a complex admixture of components is to ensure that all components are stabilised in the admixture so that they are not denatured between the point of manufacture and the point of use.

This problem is particularly prevalent wherein the detergent composition includes components which are antagonistic towards other detergent components. In this regard bleaches are case in point: typically they bring about oxidative destruction of many other detergent components. A further example is pH: often a pH which brings about stability of one component may bring about the eradication of another.

One way to address this problem is to keep the components having different storage requirements separate until their point of use. This is relatively facile when the both components are in solid form since a separate environment for the two components can easily be created. Thus cleaning powders and compressed particulate tablets can be produced which contain multiple ingredients in solid form. Additionally often the components requiring different storage environments are segregated with the composition as a further aid to prevent premature reaction.

However, certain cleaning preparations require the use of a liquid formulation. In such a case the facile separation solution cannot easily be achieved since the components are free to migrate within the liquid and will, if they come into contact, react with one another.

In this case traditionally it has been necessary to provide liquid cleaning formulations in multi-chamber packs, wherein one chamber contains one component and a second chamber contains another component, so that different storage environments are created and the components are only brought into contact at the point of use. Such twin chamber packs are expensive to manufacture and cumbersome in use, requiring an unnecessary burden of dexterity from a consumer.

It would be desirable to have a multi-component cleaning composition with high level of efficacy in use and with a high degree of stability before use.

In other fields it is recognised that treatment compositions, aside from providing the primary aim, can be harmful to a substrate, such that overly prolonged exposure to the substrate can cause damage. Here it is acknowledged that after a certain exposure time quenching of the treatment composition is advisable to prevent harm caused by over exposure of the substrate to the treatment composition.

This is particularly true of skin care compositions due to the sensitive nature of human skin and especially true of depilatory compositions.

Compositions for removing superfluous body hair are well known and are of various types. Depilatory compositions of the type which degrades the hairs comprise a depilatory compound which is able to degrade the hairs. Depilatory compounds in common use, such as potassium thioglycolate, and other such compounds having a thiol group, have a disadvantage in that they are particularly aggressive. This is of course beneficial when it comes to hair removal but can be problematic in terms of skin damage in cases of over exposure.

Furthermore depilatory compositions typically contain compounds which can irritate and even damage the skin. For example they typically contain sodium hydroxide to provide a high pH. The depilatory compositions are applied to the skin and allowed to act on the skin for a sufficient time to degrade the hairs. However, the compositions should not be allowed to act on the skin for longer than a certain time so as to reduce the irritant effect and possible damage to the skin. Although instructions are typically provided with depilatory compositions informing the user of the correct residence time, users do not always read them or follow the instructions correctly. It would therefore be desirable to have a composition which has an appropriate end-of-life indication after a suitable residence time so that a user knows when it is appropriate to remove the composition or which indicates when the composition is likely to have remained on the user's skin too long.

Thus users have to exercise a degree of care such that the unwanted hair is exposed to the depilatory composition for a sufficient amount of time such that the unwanted hair is degrade such that it can be removed without causing skin damage.

It would be desirable to have a depilatory composition with a reduced level of damage potential when in use.

According to a first aspect of the invention there is provided a method of delivering a primary active agent and a secondary active agent to a locus using a polymersome containing composition, wherein the composition comprises a plurality of polymersome vesicles containing the secondary active agent and a liquid matrix comprising the primary active agent, characterised in that the polymersome vesicles are capable of being disrupted.

Preferably the disruption mechanism is a chemical and/or mechanical disruption. Preferred disruption mechanisms include the application of mechanical shear and/or change in osmotic potential.

Preferably the method is for use in treating a substrate/surface. More preferably the method is used for treating a substrate/surface wherein the primary active agent has to be quenched after a certain time period such that it can achieve its aim on the substrate/surface without causing any damage thereto.

Thus according to a second aspect of the invention there is provided a method of delivering an active agent and a quenching agent to a surface using a polymersome containing composition, wherein the composition comprises a plurality of polymersome vesicles containing a quenching agent and a liquid matrix comprising the active agent, characterised in that the polymersome vesicles are capable of being disrupted by the application of mechanical shear.

Further according to a third aspect of the invention there is provided a composition comprising an active agent and a quenching agent, wherein the composition comprises a plurality of polymersome vesicles containing a quenching agent and a liquid matrix comprising the active agent, characterised in that the polymersome vesicles are capable of being disrupted by the application of mechanical shear.

Preferably the substrate/surface is a skin surface. It is preferred that the active agent is a depilatory agent.

We have surprisingly discovered that by using a quenching agent in a depilatory formulation the potential for skin damage for a user is vastly reduced without affecting the performance of the depilatory agent. It is postulated that this is because the quenching agent is released from the polymersome by a shearing action in use thus the active depilatory agent is de-activated (as is the potential for skin) damage.

It will be appreciated that the quenching agent is capable of reacting with the active agent so as to remove the activity of the active agent.

The depilatory agent may be any compound capable of degrading keratin. Examples of such agents are potassium thioglycolate, dithioerythritol, thioglycerol, thioglycol, thioxanthine, thiosalicylic acid, N-acetyl-L-cysteine, lipoic acid, sodium dihydrolipoate 6,8-dithioocatanoate, sodium 6,8-diothioocatanoate, a hydrogen sulphide salt, thioglycolic acid, 2-mercaptopropionic acid, 3-mercaptopropionic acid, thiomalic acid, ammonium thioglycolate, glyceryl monothioglycolate, monoethanolamine thioglycolate, monoethanolamine thioglycolic acid, diammoniumdithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glycerylmonothioglycolate, thioglycolhydrazine, keratinase, guanidine thioglycolate, calcium thioglycolate and/or cysteamine. A single compound or a mixture of two or more compounds may be used.

Preferably the depilatory agent is potassium thioglycolate.

The depilatory agent may comprise a source of alkalinity, for example an alkali metal hydroxide such as sodium hydroxide or potassium hydroxide. Desirably the pH of the depilatory agent of the present invention is at least 12, more preferably at least 12.4.

Preferably the alkali metal hydroxide is present in an amount of at least 0.001 mol/100 g of composition, preferably in an amount of at least 0.01 mol/100 g.

The composition may comprise further components such as perfumes, oils, pigments, clays, fillers such as lithium sodium is magnesium silicate, magnesium trisilicate and titanium dioxide.

In accordance with a fourth aspect of the invention there is provided a method of depilation comprising:
a. applying to the skin a composition as defined in above;
b. allowing the composition a residence time on the skin to degrade hairs;
c. applying a shear to the composition to cause the polymersomes in the composition to at least partially degrade to release quenching agent;
d. removing the composition and depilated hairs from the skin; and
e. rinsing the skin.

Preferably the residence time on the skin is from 3 to 10 minutes. Desirably the appropriate residence time is coordinated with a colour change. The composition may be removed from the skin using a spatula or scraper device.

Preferably the method is for use in cleaning a substrate/surface. More preferably the method is used for cleaning a substrate/surface wherein the activated of the primary active agent has to be complemented/augmented that it can achieve its aim on the substrate/surface.

Thus according to a fifth aspect of the invention there is provided a method of delivering a cleaning composition comprising a primary active agent and a secondary active agent to a substrate/surface, wherein the composition comprises a plurality of polymersome vesicles containing the secondary active agent and a liquid matrix comprising the primary active agent, characterised in that the polymersome vesicles are capable of being disrupted.

Further according to a sixth aspect of the invention there is provided a cleaning composition comprising a primary active agent and a secondary active agent, wherein the composition comprises a plurality of polymersome vesicles containing the secondary active agent, and a liquid matrix comprising the primary active agent, characterised in that the polymersome vesicles are capable of being disrupted.

Preferably the disruption mechanism is by change in osmotic potential (e.g. by dilution of the composition when added to a wash liquor).

Preferably the substrate/surface comprises a hard surface such as house ware (e.g. crockery, cutlery, cooking utensils and/or vessels); sanitary ware/bath ware (e.g. toilet bowls, baths, sinks, showers, taps); food preparation and/or cooking surfaces; soft surfaces such as clothing and other fabrics (e.g. carpets, wipes).

We have surprisingly discovered that by separation of the primary and secondary active agents formulations with high efficacy and yet high stability pre-use can be prepared.

Preferably the primary active agent comprises a bleach. Preferred examples of bleaches include peroxygen compounds. Suitable peroxygen compounds include hydrogen peroxide, perborates, percarbonates, persulfates, peroxy disulfates, perphosphates and the crystalline peroxyhydrates formed by reacting hydrogen peroxide with urea or alkali metal carbonate. Other examples of bleaches include per-acids such as phthalimidoperhexanoic acid (PAP).

Preferably the secondary active agent is an agent which complements the bleach and/or is one which is either antagonistic toward the bleach or detrimentally damaged by exposure to the bleach.

Preferred examples of agents which complement the bleach include bleach activators. The best known organic bleach activator of practical importance is N,N,N,N-tetraacyl ethylene diamine, normally referred to as TAED. Another well-known bleach activator is sodium benzoyl oxybenzene sulphonate normally referred to as BOBS. Yet another well-known bleach activator is decanoyl oxybenzoic acid normally referred to as DOBA Examples of other organic bleach activators are other n-acyl substituted amides, for example tetraacetyl methylene diamine; carboxylic acid anhydrides for example succinic, benzoic and phthalic anhydrides; carboxylic acid esters, for example sodium acetoxy benzene sulphonate; acetates such as glyceroltriacetate, glucose pentaacetate and xylose5 tetraacetate and acetyl salicylic acid. Where present, preferably TAED is used as the bleach activator.

Preferred examples of agents which are detrimentally damaged by exposure to the bleach include enzymes such as lipases or proteases, other enzymes such as cellulase (Carezymem™, Clazinasem™, Celluzymem™), oxidase, amylase, peroxidase may be used. The enzymes may be used together with cofactors required to promote enzymes activity, i.e. they may be used in enzymes systems, if required. It should also be understood that enzymes having mutations at various positions (e.g. enzymes engineered for performance and/or stability enhancement) are also contemplated by the invention.

### Detergent Active

The cleaning composition may contain one or more surface active agents selected from the group consisting of anionic, nonionic, cationic, ampholytic and zwitterionic surfactants or mixtures thereof. The preferred surfactant detergents for are mixtures of anionic and nonionic surfactants although it is to be understood that any surfactant may be used alone or in combination with any other surfactant or surfactants.

### Anionic Surfactant Detergents

Anionic surface active agents which may be used are those surface active compounds which contain a long chain hydrocarbon hydrophobic group in their molecular structure and a hydrophilic group, i.e. water solubilising group such as carboxylate, sulfonate or sulphate group or their corresponding acid form. The anionic surface active agents include the alkali metal (e.g. sodium and potassium) water soluble higher alkyl aryl sulphonates, alkyl sulphonates, alkyl sulphates and the alkyl poly ether sulphates. They may also include fatty acids or fatty acid soaps. One of the preferred groups of anionic surface active agents are the alkali metal, ammonium or alkanolamine salts of higher alkyl aryl sulphonates and alkali metal, ammonium or alkanolamine salts of higher alkyl sulphates. Preferred higher alkyl sulphates are those in which the alkyl groups contain 8 to 26 carbon atoms, preferably 10 to 22 carbon atoms and more preferably 12 to 18 carbon atoms. The alkyl group in the alkyl aryl sulfonate preferably contains 8 to 16 carbon atoms and more preferably 10 to 15 carbon atoms. A particularly preferred alkyl aryl sulfonate is the sodium, potassium or ethanolamine C₁₀ to C₁₆ benzene sulfonate, e.g. sodium linear dodecyl benzene sulfonate. The primary and secondary alkyl sulphates can be made by reacting long chain alpha-olefins with sulphites or bisulphites, e.g. sodium bisulfite. The alkyl sulphates can also be made by reacting long chain normal paraffin hydrocarbons with sulphur dioxide and oxygen as described in U.S. Pat. Nos. 2,503,280, 2,507,088, 3372188 and 3260741 to obtain normal or secondary higher alkyl sulphates suitable for use as surfactant detergents.

The alkyl substituent is preferably linear, i.e. normal alkyl, however, branched chain alkyl sulfonates can be employed, although they are not as good with respect to biodegradability. The alkane, i.e. alkyl, substituent may be terminally sulfonated or may be joined, for example, to the 2-carbon atom of the chain, i.e. may be a secondary sulfonate. It is understood in the art that the substituent may be joined to any carbon on the alkyl chain. The higher alkyl sulfonates can be used as the alkali metal salts, such as sodium and potassium. The preferred salts are the sodium salts. The preferred alkyl sulfonates are the C₁₀ to C₁₈, primary normal alkyl sodium and potassium sulfonates, with the C₁₀ to C₁₅ primary normal alkyl sulfonate salt being more preferred.

Normal alkyl and branched chain alkyl sulfates (e.g. primary alkyl sulfates) may be used as the anionic component.

The higher alkyl polyethoxy sulfates may be used and can be normal or branched chain alkyl and contain lower alkoxy groups which can contain two or three carbon atoms. The normal higher alkyl polyether sulfates are preferred in that they have a higher degree of biodegradability than the branched chain alkyl and the lower poly alkoxy groups are preferably ethoxy groups.

The preferred higher alkyl polyethoxy sulfates are represented by the formula: R¹—O(CH₂CH₂O)ₚ—SO₃M, where R¹ is C₈ to C₂₀ alkyl, preferably C₁₀ to C₁₈ and more preferably C₁₂ to C₁₅; p is 2 to 8, preferably 2 to 6, and more preferably 2 to 4; and M is an alkali metal, such as sodium and potassium, or an ammonium cation. The sodium and potassium salts are preferred.

A preferred higher alkyl poly ethoxylated sulfate is the sodium salt of a triethoxy C₁₂ to C₁₅ alcohol sulfate having the formula: C₁₂₋₁₅—O—(CH₂CH₂O)₃—SO₃Na

Examples of suitable alkyl ethoxy sulfates that can be used are C₁₂₋₁₅ normal or primary alkyl triethoxy sulfate, sodium salt; n-decyl diethoxy sulfate, sodium salt; C₁₂ primary alkyl diethoxy sulfate, ammonium salt; C₁₂ primary alkyl triethoxy sulfate, sodium salt; C₁₅ primary alkyl tetraethoxy sulfate, sodium salt; mixed C₁₄₋₁₅ normal primary alkyl mixed tri- and tetraethoxy sulfate, sodium salt; stearyl pentaethoxy sulfate, sodium salt; and mixed C₁₀₋₁₈ normal primary alkyl triethoxy sulfate, potassium salt.

The normal alkyl ethoxy sulfates are readily biodegradable and are preferred. The alkyl poly-lower alkoxy sulfates can be used in mixtures with each other and/or in mixtures with the above discussed higher alkyl benzenesulfonates, or alkyl sulfates.

### Nonionic Surfactant

Part of the surfactant composition may be a nonionic surfactant.

Sugar or glycoside surfactants suitable for use include those discussed in the following patents: U.S. Pat. No. 5,573,707, U.S. Pat. No. 5,562,848, U.S. Pat. No. 5,542,950, WO 96/15305, U.S. Pat. No. 5,529,122, WO 95/33036, and DE 4,234,241.

Nonionic surfactants which may be used include polyhydroxy amides as discussed in U.S. Pat. No. 5,312,954 and aldobionamides such as disclosed in U.S. Pat. No. 5,389,279.

Another class of sugar based surfactants which can be used include N-alkoxy or N-aryloxy polyhydroxy fatty acid amides discussed in WO 95/07256, WO 92/06071, and WO 92/06160. These references are incorporated by reference into the subject application. Yet another class of sugar based surfactants are sugar esters discussed in GB 2061313, GB 2048670, EP 20122 and U.S. Pat. No. 4,259,202.

As is well known, the nonionic surfactants are characterized by the presence of a hydrophobic group and an organic hydrophilic group and are typically produced by the condensation of an organic aliphatic or alkyl aromatic hydrophobic compound with ethylene oxide (hydrophilic in nature). Typical suitable nonionic surfactants are those disclosed in U.S. Pat. Nos. 4,316,812 and 3,630,929.

Usually, the nonionic surfactants are polyalkoxylated lipophiles wherein the desired hydrophile-lipophile balance is obtained from addition of a hydrophilic poly-lower alkoxy group to a lipophilic moiety. A preferred class of nonionic surfactant is the alkoxylated alkanols wherein the alkanol is of 9 to 18 carbon atoms and wherein the number of moles of alkylene oxide (of 2 or 3 carbon atoms) is from 3 to 12. Of such materials it is preferred to employ those wherein the alkanol is a fatty alcohol of 9 to 11 or 12 to 15 carbon atoms and which contain from 5 to 8 or 5 to 9 alkoxy groups per mole.

Exemplary of such compounds are those wherein the alkanol is of 10 to 15 carbon atoms and which contain 5 to 9 ethylene oxide groups per mole, e.g. Neodol 25-9 and Neodol 23-6.5, which products are made by Shell Chemical Company, Inc. The former is a condensation product of a mixture of higher fatty alcohols averaging 12 to 15 carbon atoms, with about 9 moles of ethylene oxide and the latter is a corresponding mixture wherein the carbon atoms content of the higher fatty alcohol is 12 to 13 and the number of ethylene oxide groups present averages about 6.5. The higher alcohols are primary alkanols.

Another subclass of alkoxylated surfactants which can be used contain a precise alkyl chain length rather than an alkyl chain distribution of the alkoxylated surfactants described above. Typically, these are referred to as narrow range alkoxylates. Examples of these include the Neodol-1® series of surfactants manufactured by Shell Chemical Company.

Other useful nonionics are represented by the commercially well known class of nonionics sold under the trademark Plurafac by BASF. The Plurafacs are the reaction products of a higher linear alcohol and a mixture of ethylene and propylene oxides, containing a mixed chain of ethylene oxide and propylene oxide, terminated by a hydroxyl group. Examples include C₁₃-C₁₅ fatty alcohol condensed with 6 moles ethylene oxide and 3 moles propylene oxide, C₁₃-C₁₅ fatty alcohol condensed with 7 moles propylene oxide and 4 moles ethylene oxide, C₁₃-C₁₅ fatty alcohol condensed with 5 moles propylene oxide and 10 moles ethylene oxide or mixtures of any of the above.

Another group of liquid nonionics are commercially available from Shell Chemical Company, Inc. under the Dobanol or Neodol trademark: Dobanol 91-5 is an ethoxylated C₉-C₁₁ fatty alcohol with an average of 5 moles ethylene oxide and Dobanol 25-7 is an ethoxylated C₁₂-C₁₅ fatty alcohol with an average of 7 moles ethylene oxide per mole of fatty alcohol.

### Cationic Surfactants

Many cationic surfactants are known in the art, and almost any cationic surfactant having at least one long chain alkyl group of about 10 to 24 carbon atoms is suitable. Such compounds are described in “Cationic Surfactants”, Jungermann, 1970, incorporated by reference. Specific cationic surfactants which can be used as surfactants are described in detail in U.S. Pat. No. 4,497,718.

As with the nonionic and anionic surfactants, the compositions may use cationic surfactants alone or in combination with any of the other surfactants known in the art. Of course, the compositions may contain no cationic surfactants at all.

### Amphoteric Surfactants

Ampholytic synthetic surfactants can be broadly described as derivatives of aliphatic or aliphatic derivatives of heterocyclic secondary and tertiary amines in which the aliphatic radical may be straight chain or branched and wherein one of the aliphatic substituents contains from about to 18 carbon atoms and at least one contains an anionic water-soluble group, e.g. carboxylate, sulfonate, sulfate. Examples of compounds falling within this definition are sodium 3-(dodecylamino)propionate, sodium 3-(dodecylamino)propane-1-sulfonate, sodium 2-(dodecylamino)ethyl sulfate, sodium 2-(dimethylamino)octadecanoate, disodium 3-(N-carboxymethyldodecylamino)propane 1-sulfonate, disodium octadecylimminodiacetate, sodium 1-carboxymethyl-2-undecylimidazole, and sodium N,N-bis(2-hydroxyethyl)-2-sulfato-3-dodecoxy-propylamine. Sodium 3-(dodecylamino)propane-1-sulfonate is preferred.

Zwitterionic surfactants can be broadly described as derivatives of secondary and tertiary amines, derivatives of heterocyclic secondary and tertiary amines, or derivatives of quaternary ammonium, quaternary phosphonium or tertiary sulfonium compounds. The cationic atom in the quaternary compound can be part of a heterocyclic ring. In all of these compounds there is at least one aliphatic group straight chain or branched, containing from about 3 to 18 carbon atoms and at least one aliphatic substituent containing an anionic water-solubilising group, e.g. carboxy, sulfonate, sulfate, phosphate, or phosphonate.

Specific examples of zwitterionic surfactants which may be used are set forth in U.S. Pat. No. 4,062,647.

### Builders/Electrolytes

Builders which can be used include conventional alkaline detergency builders, inorganic or organic.

As electrolyte may be used any water-soluble salt. Electrolyte may also be a detergency builder, such as the inorganic builder sodium tripolyphosphate, or it may be a non-functional electrolyte such as sodium sulphate or chloride. Preferably the inorganic builder comprises all or part of the electrolyte. That is the term electrolyte encompasses both builders and salts.

Examples of suitable inorganic alkaline detergency builders which may be used are water-soluble alkali metal phosphates, polyphosphates, borates, silicates and also carbonates. Specific examples of such salts are sodium and potassium triphosphates, pyrophosphates, orthophosphates, hexametaphosphates, tetraborates, silicates and carbonates.

Examples of suitable organic alkaline detergency builder salts are: (1) water-soluble amino polycarboxylates, e.g. sodium and potassium ethylenediaminetetraacetates, nitrilotriacetates and N-(2 hydroxyethyl)-nitrilodiacetates; (2) water-soluble salts of phytic acid, e.g. sodium and potassium phytates (see U.S. Pat. No. 2,379,942); (3) water-soluble polyphosphonates, including specifically, sodium, potassium and lithium salts of ethane-1-hydroxy-1,1-diphosphonic acid; sodium, potassium and lithium salts of methylene diphosphonic acid; sodium, potassium and lithium salts of ethylene diphosphonic acid; and sodium, potassium and lithium salts of ethane-1,1,2-triphosphonic acid. Other examples include the alkali metal salts of ethane-2-carboxy-1,1-diphosphonic acid hydroxymethanediphosphonic acid, carboxyldiphosphonic acid, ethane-1-hydroxy-1,1,2-triphosphonic acid, ethane-2-hydroxy-1,1,2-triphosphonic acid, propane-1,1,3,3-tetraphosphonic acid, propane-1,1,2,3-tetraphosphonic acid, and propane-1,2,2,3-tetraphosphonic acid; (4) water-soluble salts of polycarboxylate polymers and copolymers as described in U.S. Pat. No. 3,308,067.

In addition, polycarboxylate builders can be used satisfactorily, including water-soluble salts of mellitic acid, citric acid, and carboxymethyloxysuccinic acid, salts of polymers of itaconic acid and maleic acid, tartrate monosuccinate, tartrate disuccinate and mixtures thereof (TMS/TDS).

### Optional Ingredients

A number of other optional ingredients may be used.

Alkalinity buffers which may be added to the compositions include monoethanolamine, triethanolamine, borax and the like.

In addition, various other detergent additives or adjuvants may be present in the detergent product to give it additional desired properties, either of functional or aesthetic nature.

There also may be included in the formulation, minor amounts of soil suspending or anti-redeposition agents, e.g. polyvinyl alcohol, fatty amides, sodium carboxymethyl cellulose, hydroxy-propyl methyl cellulose. Preferred anti-redeposition agents include Alcosperse 725™ and sodium carboxylmethyl cellulose having a 2:1 ratio of CM/MC which is sold under the tradename Relatin DM 4050

Optical brighteners for cotton, polyamide and polyester fabrics can be used. Suitable optical brighteners include Tinopal LMS-X, Tinopal UNPA-GX, stilbene, triazole and benzidine sulfone compositions, especially sulfonated substituted triazinyl stilbene, sulfonated naphthotriazole stilbene, benzidene sulfone, etc., most preferred are stilbene and triazole combinations. A preferred brightener is Stilbene Brightener N4 which is a dimorpholine dianilino stilbene sulfonate.

Anti-foam agents, e.g. silicon compounds, such as Silicane L 7604, can also be added in small effective amounts.

Bactericides, e.g. tetrachlorosalicylanilide and hexachlorophene, fungicides, dyes, pigments (water dispersible), preservatives, e.g. formalin, ultraviolet absorbers, anti-yellowing agents, such as sodium carboxymethyl cellulose, pH modifiers and pH buffers, colour safe bleaches, perfume and dyes and bluing agents such as Iragon Blue L2D, Detergent Blue 472/572 and ultramarine blue can be used.

Also, soil release polymers and cationic softening agents may be used.

“Polymersomes” are vesicles, which are assembled from synthetic multi-block polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self assembly,” a spontaneous, entropy-driven process of preparing a closed semi-permeable membrane.

Because of the perselectivity of the bilayer, materials may be “encapsulated” in the aqueous interior (lumen) or intercalated into the hydrophobic membrane core of the polymersome vesicle, forming a “loaded polymersome.” Numerous technologies can be developed from such vesicles, owing to the numerous unique features of the bilayer membrane and the broad availability of super-amphiphiles, such as diblock, triblock, or other multi-block copolymers.

The synthetic polymersome membrane can exchange material with the “bulk,” i.e., the solution surrounding the vesicles. Each component in the bulk has a partition coefficient, meaning it has a certain probability of staying in the bulk, as well as a probability of remaining in the membrane. Conditions can be predetermined so that the partition coefficient of a selected type of molecule will be much higher within a vesicle's membrane, thereby permitting the polymersome to decrease the concentration of a molecule, such as cholesterol, in the bulk. In a preferred embodiment, phospholipid molecules have been shown to incorporate within polymersome membranes by the simple addition of the phospholipid molecules to the bulk. In the alternative, polymersomes can be formed with a selected molecule, such as a hormone, incorporated within the membrane, so that by controlling the partition coefficient, the molecule will be released into the bulk when the polymersome arrives at a destination having a higher partition coefficient.

Polymersomes may be formed from synthetic, amphiphilic copolymers. An “amphiphilic” substance is one containing both polar (water-soluble) and hydrophobic (water-insoluble) groups. “Polymers” are macromolecules comprising connected monomeric units. The monomeric units may be of a single type (homogeneous), or a variety of types (heterogeneous). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains). For example, in polyethylene glycol (PEG), which is a polymer of ethylene oxide (EO), the chain lengths which, when covalently attached to a phospholipid, optimize the circulation life of a liposome, is known to be in the approximate range of 34-114 covalently linked monomers (EO34 to EO114).

The preferred class of polymer selected to prepare the polymersomes is the “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

In the “melt” (pure polymer), a diblock copolymer may form complex structures as dictated by the interaction between the chemical identities in each segment and the molecular weight. The interaction between chemical groups in each block is given by the mixing parameter or Flory interaction parameter, [chi], which provides a measure of the energetic cost of placing a monomer of A next to a monomer of B. Generally, the segregation of polymers into different ordered structures in the melt is controlled by the magnitude of [chi]N, where N is proportional to molecular weight. For example, the tendency to form lamellar phases with block copolymers in the melt increases as [chi]N increases above a threshold value of approximately 10.

A linear diblock copolymer of the form A-B can form a variety of different structures. In either pure solution (the melt) or diluted into a solvent, the relative preferences of the A and B blocks for each other, as well as the solvent (if present) will dictate the ordering of the polymer material. In the melt, numerous structural phases have been seen for simple AB diblock copolymers.

To form a stable membrane in water, the absolute minimum requisite molecular weight for an amphiphile must exceed that of methanol HOCH₃, which is undoubtedly the smallest canonical amphiphile, with one end polar (HO—) and the other end hydrophobic (—CH₃). Formation of a stable lamellar phase more precisely requires an amphiphile with a hydrophilic group whose projected area, when viewed along the membrane's normal, is approximately equal to the volume divided by the maximum dimension of the hydrophobic portion of the amphiphile (Israelachvili, in Intermolecular and Surface Forces, 2 less than nd ed., Pt3 (Academic Press, New York) 1995).

The most common lamellae-forming amphiphiles also have a hydrophilic volume fraction between 20 and 50 percent. Such molecules form, in aqueous solutions, bilayer membranes with hydrophobic cores never more than a few nanometers in thickness. The present invention relates to polymersomes with all super-amphiphilic molecules which have hydrophilic block fractions within the range of 20-50 percent by volume and which can achieve a capsular state. The ability of amphiphilic and super-amphiphilic molecules to self-assemble can be largely assessed, without undue experimentation, by suspending the synthetic super-amphiphile in aqueous solution and looking for lamellar and vesicular structures as judged by simple observation under any basic optical microscope or through the scattering of light.

For typical phospholipids with two acyl chains, temperature can affect the stability of the thin lamellar structures, in part, by determining the volume of the hydrophobic portion. In addition, the strength of the hydrophobic interaction, which drives self-assembly and is required to maintain membrane stability, is generally recognized as rapidly decreasing for temperatures above approximately 50° C. Such vesicles generally are not able to retain their contents for any significant length of time under conditions of boiling water.

Upper limits on the molecular weight of synthetic amphiphiles which form single component, encapsulating membranes clearly exceed the many kilodalton range, as concluded from the work of Discher et al., (1999).

Block copolymers with molecular weights ranging from about 2 to 10 kilograms per mole can be synthesized and made into vesicles when the hydrophobic volume fraction is between about 20 percent and 50 percent. Diblocks containing polybutadiene are prepared, for example, from the polymerization of butadiene in cyclohexane at 40[deg.] C. using sec-butyllithium as the initiator. Microstructure can be adjusted through the use of various polar modifiers. For example, pure cyclohexane yields 93 percent 1.4 and 7 percent 1.2 addition, while the addition of THF (50 parts per L1) leads to 90 percent 1.2 repeat units. The reaction may be terminated with, for example, ethyleneoxide, which does not propagate with a lithium counterion and HCl, leading to a monofunctional alcohol. This PB-OH intermediate, when hydrogenated over a palladium (Pd) support catalyst, produces PEE-OH. Reduction of this species with potassium naphthalide, followed by the subsequent addition of a measured quantity of ethylene oxide, results in the PEO-PEE diblock copolymer. Many variations on this method, as well as alternative methods of synthesis of diblock copolymers are known in the art; however, this particular preferred method is provided by example, and one of ordinary skill in the art would be able to prepare any selected diblock copolymer.

For example, if PB-PEO diblock copolymers were selected, the synthesis of PB-PEO differs from the previous scheme by a single step, as would be understood by the practitioner. The step by which PB-OH is hydrogenated over palladium to form PEO-OH is omitted. Instead, the PB-OH intermediate is prepared, then it is reduced, for example, using potassium naphthalide, and converted to PB-PEO by the subsequent addition of ethylene oxide.

In yet another example, triblock copolymers having a PEO end group can also form polymersomes using similar techniques. Various combinations are possible comprising, e.g., polyethylene, polyethylethylene, polystyrene, polybutadiene, and the like. For example, a polystyrene (PS)-PB-PEO polymer can be prepared by the sequential addition of styrene and butadiene in cyclohexane with hydroxyl functionalization, re-initiation and polymerization. PB-PEE-PEO results from the two-step polymerization of butadiene, first in cyclohexane, then in the presence of THF, hydrolyl functionalization, selective catalytic hydrogenation of the 1.2 PB units, and the addition of the PEO block.

A plethora of molecular variables can be altered with these illustrative polymers, hence a wide variety of material properties are available for the preparation of the polymersomes. ABC triblocks can range from molecular weights of 3,000 to at least 30,000 g/mol. Hydrophilic compositions should range from 20-50 percent in volume fraction, which will favor vesicle formation. The molecular weights must be high enough to ensure hydrophobic block segregation to the membrane core. The Flory interaction parameter between water and the chosen hydrophobic block should be high enough to ensure said segregation. Symmetry can range from symmetric ABC triblock copolymers (where A and C are of the same molecular weight) to highly asymmetric triblock copolymers (where, for example, the C block is small, and the A and B blocks are of equal length).

The polymersomes are preferably based on A PBd-PEO copolymer. Alternative polymers include poly(hexyl methacrylate)-block-poly[2-(dimethylamino)ethyl methacrylate] (PHMA-PDMA), poly(hexyl methacrylate)-block-poly(methacrylic acid) (PHMA-PMAA), poly(butyl methacrylate)-block-poly(methacrylic acid) (PBMA-PMAA), poly(ethylene oxide)-block-poly(hexyl methacrylate) (PEO-PHMA), poly(butyl methacrylate)-block-poly[2-(dimethylamino)ethyl methacrylate (PBMA-PDMA), poly(hexyl methacrylate)-block-poly[2-(dimethylamino)ethyl methacrylate (PHMA-PDMA), poly(butyl methacrylate)-block-Poly(ethylene oxide) (PBMA-PEO).

Generally (following synthesis) such a polymer is used to form polymersomes (vesicles).

A preferred method of polymersome synthesis is below:—

### Polymer Synthesis

10 g of the PBd-PEO block copolymer was synthesised via anionic polymerisation. Ion exchange between PBd-OH 1,4 (90-95%) in the presence of α-ethylstyrene and potassium resulted in the formation of PBd-O⁻K⁺ which could then react with ethylene oxide. The ethylene oxide was added at 0° C. and then heated to 45° C. for 72 h with stirring to grow the ethylene oxide block of the polymer. The reaction was terminated with methanol in the presence of acid. The reaction scheme is shown below.

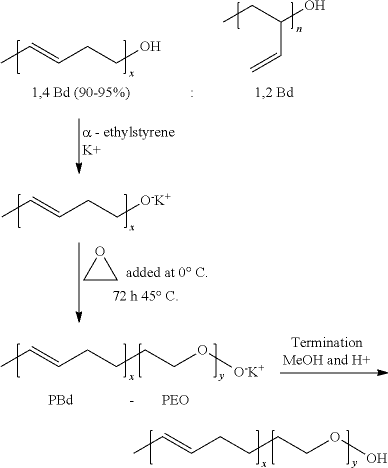

### Vesicle Formation

Porphyrine loaded vesicles of PBd-PEO were prepared as follows: First 10 mg of the PBd-PEO polymer (5000, 1500 gmol⁻¹ respectively) was dissolved in 1 ml chloroform in a sample vial. The solvent was removed under a stream of nitrogen before the vial was placed under vacuum overnight to create a film of the polymer on the sample vial surface. A solution of porphyrine (10 ml, 0.1 mM in distilled water) was made and adjusted to pH 2 using 0.1M hydrochloric acid (HCl) and 0.1M sodium hydroxide (NaOH) solutions. This resulted in a colour change from a red to a green solution. The polymer film was rehydrated by adding 5 ml of the porphyrine solution with stirring and was then sonicated for 30 min. The solution was stirred vigorously for 72 h at 50° C. to obtain porphyrine loaded polymer vesicles. The solution was then adjusted to pH 7 before it was loaded onto a sepharose 4B (Aldrich) column (15×1.5 cm) with a distilled water eluent. Fractions were collected according to the colour of the solution leaving the column. The first fraction was yellow (vesicles), it was eventually followed by a green fraction (oxidised porphyrine) which was closely followed by a red fraction (free porphyrine). The column had the combined effect of removing free porphyrine and limiting the size distribution of the vesicles. The solution was adjusted to pH 12 making a strongly alkaline supernatant.

### Scale-Up

A scale-up process was carried out as follows: First 3.5 g of the PBd-PEO polymer was dissolved in 350 ml chloroform in a 1 L round bottomed flask. The solvent was removed via rotary evaporation and placed under vacuum overnight to create a film of the polymer on the flask surface. A solution of porphyrine (Aldrich, 700 ml, 0.5 mM in distilled water) was made and adjusted to pH 2 using 1M HCl and 1M NaOH solutions. The polymer film was rehydrated by adding the porphyrine solution with stirring. These vesicles were made at a higher concentration in order to try and obtain a more concentrated solution of vesicles. The solution was stirred vigorously for 7 days at 50° C. It was then adjusted to pH 7 before loading the solution onto a sepharose 4B (Aldrich) column (15×6 cm) with a distilled water eluent. Fractions were collected according to the colour of the solution leaving the column. The solution was then adjusted to pH 12 making a strongly alkaline supernatant.

### Mechanical Measurements: Scale-Up

The pH was measured after shearing the vesicles. It had lowered from 12 to 11.2 as the maximum shear experienced by the vesicles was increased. The observed decrease in the pH indicates that acid is gradually being released as the shear rate and extent of shear increases. This result is consistent with the idea that shearing is causing minor disruptions of the vesicle shell. Pores in the vesicles are continuously forming and “healing” allowing the acid to leak into the supernatant.

| Rate (s⁻¹) | Stress (Pa) | Time (s) | pH |
|---|---|---|---|
| Control | — | — | 12.15 |
| 250 | 0.58 | 90 | 11.64 |
| 1000 | 3.3 | 150 | 11.67 |
| 1500 | 6.4 | 185 | 11.4 |
| 2600 | 9.8 | 255 | 11.20 |

Salt was then added in amounts corresponding to those used in the previous research (50 mg/ml NaCl). After 1 day the pH had reduced from 9.7 to 8.8, an overall change from 12 to 8.8, which shows a significant amount of acid had been taken up by the vesicles. Mechanical stirring was attempted but at the rates used no effect was observed on the pH of the vesicle solution.

By the time these subsequent measurements were made, however, the pH of the solution had dropped to 9.7 which indicated that the vesicles had started to leak acid into the basic environment; therefore these results should be received with caution. This could be due to the scale-up process allowing the formation of multilayer vesicles which are less stable than single layer vesicles as they are in higher energy minima. This problem could be reduced by sonicating the vesicles after the polymer has been dissolved to break and reform the vesicle structure (FIG. 21), this could also allow the vesicles to take up more of the acidic solution.

### Conclusions

A PBd-PEO copolymer was synthesised and used to form vesicles in water that could encapsulate an acidic solution. The pH of the supernatant was then increased significantly and a kinetically stable partition of encapsulated, fluorescent solution at pH 2 and a pH-metered supernatant at pH 12 could be stored before subjecting vesicles to shear.

Shearing by rheometry yielded promising results: although pH changes were small. Crucially, sensitivity of indicated pH to mechanical shear was observed for an experimental batch, a confirmation batch and a scale-up attempt, though it appears likely that the different technique used in scaling up gives rise to an instability in the vesicles that allows them to leak into the supernatant.

1. A method of delivering active agents to a locus comprising:
delivering a primary active agent and a secondary active agent to a locus using a polymersome containing composition;
wherein the composition comprises a plurality of polymersome vesicles containing the secondary active agent and a liquid matrix comprising the primary active agent; and
wherein the polymersome vesicles are capable of being disrupted by a disruption mechanism.

2. The method according to claim 1, wherein the disruption mechanism comprises one or more of a chemical and mechanical disruption.

3. The method according to claim 2, wherein the disruption mechanism comprises the application one or more of mechanical shear and change in osmotic potential.

4. (canceled)

5. A method of treating a substrate/surface comprising the method of claim 1.

6. The method according to claim 5, further comprising quenching the primary active agent after a certain time period.

7. The method according to claim 6, wherein the second active comprises a quenching agent.

8. The method according to claim 7, wherein the primary active agent has a high pH.

9. The method according to claim 7, wherein the quenching agent has a low pH.

10. The method according to claim 9, wherein the quenching agent comprises an acid.

11. The method according to claim 5, wherein the substrate/surface is a skin surface.

12. The method according to claim 11, wherein the primary active agent comprises a depilatory agent.

13. The method according to claim 12, wherein the depilatory agent comprises alkaline potassium thioglycolate.

14. The method according to claim 1, wherein the polymersome comprises a vesicle formed from an amphilic di-block copolymer.

15. The method according to claim 1, wherein the concentration of polymersome is 0.5-1% by weight.

16. The method according to claim 1, wherein the polymersome is prepared by one or more of extrusion and sonification.

17. The method according to claim 1, wherein the disruption mechanism comprises the application of mechanical shear; and
wherein the amount of shear is from 0.5-50 Pa.

18. The method according to claim 1, wherein the disruption mechanism comprises the application of mechanical shear; and
wherein the time needed for shear is less than 10 minutes.

19. The method according to claim 1, wherein the polymersome comprises a vesicle formed from an admixture of polybutadiene (PBd) and polyethylene oxide (PEO) copolymers;
wherein the concentration of polymersome is 0.5-1% by weight;
wherein the disruption mechanism comprises the application of mechanical shear;
wherein the amount of shear is from 0.5-10 Pa; and
wherein the time needed for shear is less than 3 minutes.

20. A composition comprising:
an active agent; and
a quenching agent;
wherein a plurality of polymersome vesicles contains the quenching agent and a liquid matrix comprising the active agent; and
wherein the polymersome vesicles are capable of being disrupted by the application of mechanical shear.
